(19) United States
(12) Patent Application Publication
(10) Pub. No.: US 2009/0012542 A1
(43) Pub. Date: Jan. 8, 2009

(54) SATIATION DEVICES AND METHODS FOR CONTROLLING OBESITY

(75) Inventors: Colleen Stack N'diaye, Hillsborough, NC (US); Geoffrey A. Orth, Sebastopol, CA (US); Jeffrey A. Smith, Petaluma, CA (US); Michael S. Williams, Santa Rosa, CA (US)

Correspondence Address: MYERS BIGEL SIBLEY & SAJOVEC, PO BOX 37428, RALEIGH, NC, 27627, US

(73) Assignee: SyneCor, LLC

(21) Appl. No.: 12/144,970

(22) Filed: Jun. 24, 2008

Related U.S. Application Data
(60) Provisional application No. 60/958,122, filed on Jul. 3, 2007.

(51) Int. Cl. A61B 17/12

(52) U.S. Cl. 606/153

(57) ABSTRACT

Satiation devices for controlling obesity and methods of implanting same are configured to create a small satiation pouch in the proximal portion of the stomach with a narrow passage leading into the lower portion of the stomach. The small satiation pouch is configured to collect a small amount of masticated food from the esophagus and the narrow passage delays emptying of the food from the satiation pouch into the larger part of the stomach, thereby causing a feeling of fullness.

### RELATED APPLICATION

This application claims the benefit of and priority to U.S. Provisional Patent Application No. 60/958,122, filed Jul. 3, 2007, the disclosure of which is incorporated herein by reference as if set forth in its entirety.

### FIELD OF THE INVENTION

The present invention relates generally to the field of devices and methods for achieving weight loss in humans, and specifically to the use of devices implantable within patients for controlling feelings of hunger.

### BACKGROUND OF THE INVENTION

Obesity is defined as having excess adipose tissue (fat). The amount of adipose tissue correlates well with Body Mass Index (BMI), which is calculated as body weight in kilograms divided by height in meters squared (BMI=kg/m²). According to the National Institutes of Health (NIH), the normal range of BMI is 18.5-24.9. Overweight is defined as a BMI of 25-29.9, class I obesity as a BMI of 30-34.9, class II obesity as a BMI of 35-39.9, and class III (extreme) obesity as a BMI >40. About 65% of the U.S. population is overweight, and about 30.4% are obese.

Obesity significantly increases both morbidity and mortality. Important disorders associated with obesity are hypertension, hyperlipidemia, coronary artery disease, diabetes mellitus type II, degenerative joint disease, and psychosocial disability. 60% of the U.S. obese population has metabolic syndrome. Obesity also increases the risk of certain cancers (colon, rectum, prostate, uterus, biliary tract, breast, and ovary), thromboembolic disorders, diseases of the alimentary tract (gastro-esophageal reflux disease (GERD), gallstone disease), surgical and obstetric complications, pulmonary dysfunction, and endocrine abnormalities. Upper body obesity (excess adipose tissue around waist or flank) is associated with more health complications than lower body obesity (excess adipose tissue around the thighs and buttocks).

Obesity is often caused by a sedentary lifestyle in combination with excess caloric intake, but as much as 40-70% of obesity may be caused by or primarily influenced by genetic factors. Obesity is often treated with multidisciplinary therapy programs that combine low-calorie diets, behavior modification, aerobic exercise, and psychosocial support. Using conventional techniques such as these, only 20% of patients will achieve a 20 lb. weight loss and maintain it for 2 years. Only 5% will achieve a 40 lb. weight loss and maintain it for 2 years. FDA-approved medications to treat obesity include Sibutramine (blocks uptake of serotonin and norepinephrine in CNS) and Orlistat (decreases fat absorption in the gastrointestinal (GI) tract). Long-term benefits of these medications in treating obesity and reducing associated morbidity and mortality have not yet been well-characterized.

Hiatal hernia may occur in obese patients. Hiatal hernia displaces the lower esophageal sphincter (LES) above the diaphragm, thus decreasing its competency and contributing to reflux. Hiatal hernia also delays clearance of gastric content, promoting more severe esophagitis, particularly Barrett's esophagus. Refluxed gastric content is cleared by esophageal peristalsis, neutralization by swallowed saliva (pH >6), and gravity.

Among patients with severe obesity, bariatric surgery is an increasingly popular option. According to a National Institute of Health (NIH) consensus panel, surgery may be an appropriate option in patients with a BMI >40, or in patients with a BMI >35 and with obesity-related complications. The roux-en-Y gastric bypass (GBP) operation is commonly performed in the U.S and is often performed laparoscopically. Patients who undergo GBP often lose close to 50% of their original body weight. 40% of patients who undergo GBP experience complications including peritonitis, abdominal wall hernias, staple line disruption, gallstones, neuropathy, marginal ulcers, thromboembolic disease, infection, stomal stenosis, nutritional symptoms, and various GI manifestations such as dumping syndrome. Mortality rates within 30 days are low however at 0-1%. Adjustable gastric banding (Lap-Band or Swedish Band), a purely restrictive procedure, can also be performed laparoscopically. Other bariatric surgery procedures include vertical banding gastroplasty, biliopancreatic diversion, and mini-gastric bypass. Sleeve gastrectomy is a surgical weight loss procedure where the size of the stomach is reduced to about 35% of the original size by surgical removal of a large portion of the stomach, following the major curve. The open edges are then attached together (often with surgical staples) to form a sleeve or tube with a “banana” shape. The procedure permanently reduces the size of the stomach. The procedure is performed laparoscopically and is not reversible. Known complications are present with each of these procedures and more successful options are desired.

### SUMMARY

In view of the above discussion, satiation devices for controlling obesity and methods of implanting same are provided. These devices are configured to create a small satiation pouch in the proximal portion of the stomach with a narrow passage leading into the lower portion of the stomach. The small satiation pouch is configured to collect a small amount of masticated food from the esophagus and the narrow passage delays emptying of the food from the satiation pouch into the larger part of the stomach, thereby causing a feeling of fullness.

According to some embodiments of the present invention, a satiation device includes a plurality of mating sections that are implanted and joined together around a portion of a stomach. Each mating section includes a proximal end portion with an arcuate interior opening, a distal end portion with an arcuate interior opening, and a plurality of elongated members extending from the proximal end portion to the distal end portion. The mating sections are configured to surround a portion of a stomach below the gastro-esophageal junction thereof and to be secured together in abutting relationship to form a chamber with proximal and distal orifices. The stomach portion within the chamber forms a satiation pouch therein to receive food ingested by the patient. The distal orifice has a restrictive configuration so as to cause a narrow passage in a portion of the stomach between the satiation pouch and the lower portion of the stomach. The proximal end portion of each mating section includes a flange portion configured to be attached to a body cavity wall, such as the diaphragm. In some embodiments, the mating sections are mirror-image halves. In some embodiments, the mating sections include three or more sections.

In some embodiments, the size of the distal orifice may be adjustable. For example, the distal orifice may be adjustable during implantation. In some embodiments, the size of the distal orifice may be adjusted remotely (i.e., transabdominally) after implantation and in various ways (e.g., mechanically, magnetically, pneumatically, hydraulically, telemetrically using RF energy or ultrasound, etc.).

The mating sections may be secured together in various ways. In some embodiments, the mating sections are secured together via a bio-compatible adhesive. In some embodiments, the mating sections are secured together via one or more snap connectors in the proximal end portions and/or distal end portions. In some embodiments, the mating sections are secured together via one or more fasteners (e.g., screws, bolts, rivets, clips, t-bar connectors, sutures, etc.).

The chamber formed by the elongated members has a generally globe-shaped configuration and may be configured such that a satiation pouch formed therein has a volume of between about 2 cubic centimeters (cc) and about 300 cc. In some embodiments, the chamber is configured such that a satiation pouch formed therein has a volume of between about 10 cc and about 30 cc. In some embodiments, the elongated members have a flexible, rope-like configuration. In some embodiments, the elongated members have an arcuate, rigid configuration.

In some embodiments, the mating sections are hingedly connected at respective proximal end portions and/or distal end portions to facilitate implantation within a patient and to eliminate implantation of separate components.

A method of forming a satiation pouch in an upper portion of a stomach of a patient, according to some embodiments of the present invention, includes securing a plurality of mating sections together around a portion of a stomach, and attaching a proximal end portion thereof to a body cavity wall via, for example, surgical clips, sutures, staples, and/or adhesive. Each mating section includes a proximal end portion with an arcuate interior opening, a distal end portion with an arcuate interior opening, and a plurality of elongated members extending from the proximal end portion to the distal end portion. The mating sections form a chamber with proximal and distal orifices, and the stomach portion within the chamber forms a satiation pouch. In some embodiments, the mating sections are secured together such that the proximal end portions of the mating sections are positioned below the gastro-esophageal junction.

In some embodiments, the mating sections are secured together via one or more snap connectors in the proximal end portions and/or via one or more snap connectors in the distal end portions. In some embodiments, the mating sections are secured together at the proximal and/or distal end portions via a biocompatible adhesive. In some embodiments, the mating sections are secured together via one or more fasteners.

In some embodiments, the mating sections are secured together around a portion of a stomach via a laparoscopy tool. In other embodiments, the mating sections are secured together around a portion of a stomach via an endoscope. In other embodiments, the mating sections are secured together around a portion of a stomach via a NOTES (Natural Orifice Translumenal Endoscopic Surgery) device inserted down the esophagus of a patient.

According to other embodiments of the present invention, a satiation device that is configured to be positioned around a portion of a stomach of a patient so as to create a satiation pouch includes a proximal end portion with an arcuate interior opening, a distal end portion with an arcuate interior opening, and a plurality of elongated members extending from the proximal end portion to the distal end portion. The proximal and distal end portions are configured to partially surround the stomach to form a chamber with proximal and distal orifices. The stomach portion within the chamber forms a satiation pouch therein to receive food ingested by the patient. The distal orifice has a restrictive configuration so as to cause a narrow passage in a portion of the stomach between the satiation pouch and the lower portion of the stomach. The proximal end portion includes a flange configured to be attached to a body cavity wall.

The chamber formed by the elongated members has a generally globe-shaped configuration and may be configured such that the satiation pouch has a volume of between about 2 cc and about 300 cc. In some embodiments, the chamber is configured such that the satiation pouch has a volume of between about 10 cc and about 30 cc. In some embodiments, the elongated members have a flexible, rope-like configuration. In other embodiments, the elongated members have an arcuate, rigid configuration.

A method of forming a satiation pouch in an upper portion of a stomach of a patient, according to some embodiments of the present invention, includes implanting a unitary device around a portion of the stomach below the gastro-esophageal junction thereof, and attaching a proximal end portion of the device to a body cavity wall of the patient. The unitary device includes a proximal end portion with an arcuate interior opening, a distal end portion with an arcuate interior opening, and a plurality of elongated members extending from the proximal end portion to the distal end portion that form a chamber with proximal and distal orifices. The unitary device has a slit that allows the device to be expanded and wrapped around the stomach. The stomach portion within the chamber forms a satiation pouch therein to receive food ingested by the patient. The distal orifice has a restrictive configuration so as to cause a narrow passage in a portion of the stomach.

In some embodiments, the unitary satiation device is implanted around a portion of a stomach via a laparoscopy tool. In other embodiments, the unitary satiation device is implanted around a portion of a stomach via an endoscope. In some embodiments, the unitary satiation device is implanted around a portion of a stomach via a NOTES device inserted down the esophagus of a patient.

Satiation devices, according to some embodiments of the present invention, may be configured to be removed from a patient. For example, mating sections may be joined together such that they can be separated from each other at a later point in time. In some embodiments of the present invention, satiation devices may be designed to be permanently implanted within a patient.

Satiation devices and methods of implanting same, according to embodiments of the present invention, are significantly simpler than conventional bariatric devices and methods. Moreover, embodiments of the present invention are configured to remain stable, and because the satiation devices are attached to the diaphragm instead of to the stomach, erosion through the GI (gastro-intestinal) tissue can be eliminated. Satiation devices, according to embodiments of the present invention may also simultaneously treat GERD and hiatal hernia, which commonly affect obese patients.

Satiation devices and methods of implanting same, according to embodiments of the present invention, are particularly effective in the treatment of hiatal hernia. Each of the various satiation devices described herein act as a lock to prevent hiatal hernia. Moreover, the flange at the proximal end of the various satiation devices described herein, and which are configured to be attached to the diaphragm of a patient, help prevent and/or repair the occurrence of hiatal hernia.

### BRIEF DESCRIPTION OF THE DRAWINGS

The accompanying drawings, which form a part of the specification, illustrate embodiments of the present invention. The drawings and description together serve to fully explain the invention.

FIG. 1 is a schematic illustration of a human stomach and a portion of the small intestine.

FIG. 2 is a perspective view of a satiation device for controlling obesity, according to some embodiments of the present invention, implanted around a portion of a stomach.

FIG. 3 is a side view of the satiation device of FIG. 2.

FIG. 4A is a view of the distal end of the satiation device of FIG. 3 taken along lines 4A-4A.

FIG. 4B is a view of the proximal end of the satiation device of FIG. 3 taken along lines 4B-4B.

FIG. 5 is a side view of one of the mating sections of the satiation device of FIG. 3.

FIG. 6A illustrates the mating section of FIG. 3 turned 90° about a vertical axis.

FIG. 6B is a view of the distal end of the mating section of FIG. 6A taken along lines 6B-6B.

FIG. 6C is a view of the proximal end of the mating section of FIG. 6A taken along lines 6C-6C.

FIG. 7 is a perspective view of a satiation device for controlling obesity, according to other embodiments of the present invention, implanted around a portion of a stomach.

FIG. 8 is a side view of the satiation device of FIG. 7.

FIG. 9A is a top perspective view of the satiation device of FIG. 8.

FIG. 9B is a view of the distal end of the satiation device of FIG. 8 taken along lines 9B-9B.

FIG. 10 is a side view of one of the mating sections of the satiation device of FIG. 8.

FIG. 11A is a view of the proximal end of the mating section of FIG. 10 taken along lines 11A-11A.

FIG. 11B is a view of the distal end of the mating section of FIG. 10 taken along lines 11B-11B.

FIG. 12 is a perspective view of a satiation device for controlling obesity, according to other embodiments of the present invention, implanted around a portion of a stomach.

FIG. 13 is a side view of the satiation device of FIG. 12.

FIG. 14A is a view of the proximal end of the satiation device of FIG. 13 taken along lines 14A-14A.

FIG. 14B is a view of the distal end of the satiation device of FIG. 13 taken along lines 14B-14B.

FIG. 15 is a side view of one of the mating sections of the satiation device of FIG. 13.

FIG. 16A is a view of the proximal end of the mating section of FIG. 15 taken along lines 16A-16A.

FIG. 16B is a view of the distal end of the mating section of FIG. 15 taken along lines 16B-16B.

FIG. 17A is a side view of the satiation device of FIG. 2 illustrating the mating sections hinged together along respective adjacent elongated members.

FIG. 17B is an enlarged partial view of the hinge in FIG. 17A.

FIG. 18A is a perspective view of the satiation device of FIG. 2 illustrating the mating sections hinged together along respective proximal and distal end portions.

FIG. 18B is an enlarged partial view of the hinge at the proximal end portion in FIG. 17A.

FIG. 19 illustrates the implantation of the satiation device of FIG. 2 around a portion of a stomach via a NOTES device inserted down the esophagus of a patient.

FIG. 20 is a perspective view of a satiation device for controlling obesity, according to some embodiments of the present invention, implanted around a stomach.

FIG. 21 is a side view of the satiation device of FIG. 20.

FIG. 22 is a partial perspective view of the satiation device of FIG. 20 illustrating the flange and slit.

FIG. 23 is a top plan view of the satiation device of FIG. 20 illustrating the flange and slit.

FIG. 24 is a cross-sectional view of the satiation device of FIG. 20 illustrating the housing, a slit edge, and the opening therein.

FIG. 25 is a cross-sectional view of a satiation device according to other embodiments of the present invention and illustrating a sizing balloon within the housing.

FIG. 26 is a plan view of the distal portion of the satiation device of FIG. 20.

FIG. 27 is a plan view of the distal portion of the satiation device of FIG. 25.

FIG. 28 is a perspective view of a satiation device for controlling obesity, according to other embodiments of the present invention, implanted around a stomach.

FIG. 29 is a side view of a satiation device for controlling obesity, according to other embodiments of the present invention.

### DETAILED DESCRIPTION

While the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention as defined by the claims. Like reference numbers signify like elements throughout the description of the figures.

As used herein, the singular forms “a,” “an,” and “the” are intended to include the plural forms as well, unless expressly stated otherwise. It should be further understood that the terms “comprises” and/or “comprising” when used in this specification are taken to specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the drawings, the thickness of lines, layers and regions may be exaggerated for clarity. It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of a device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of “over” and “under”. A device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a “first” element, component, region, layer or section discussed below could also be termed a “second” element, component, region, layer or section without departing from the teachings of the present invention.

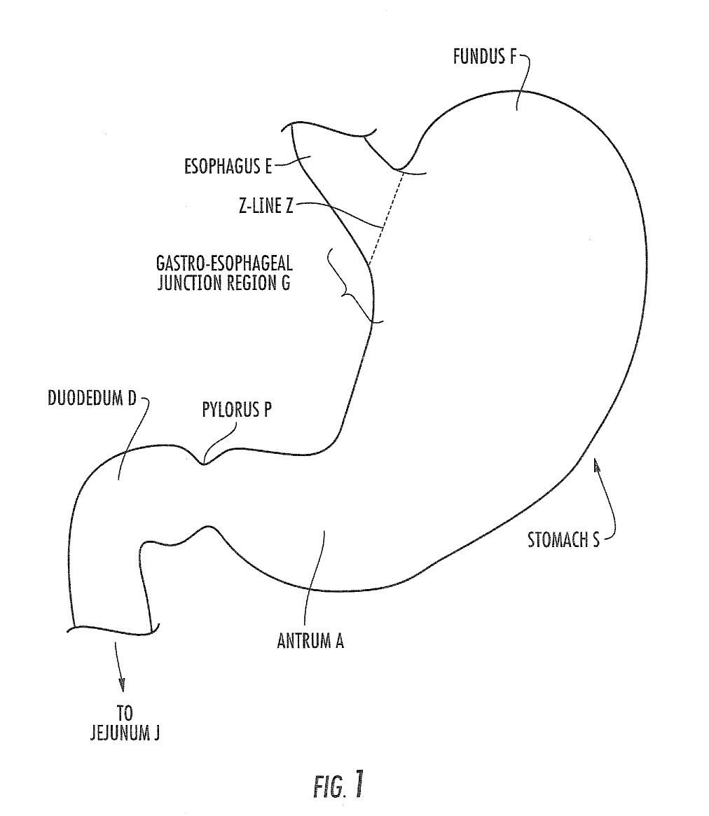

An anatomical view of a human stomach S and associated features is shown in FIG. 1. The esophagus E delivers food from the mouth to the stomach S. The z-line or gastro-esophageal junction Z is the irregularly-shaped border between the thin tissue of the esophagus and the thicker tissue of the stomach wall. The gastro-esophageal junction region G is the region encompassing the distal portion of the esophagus E, the z-line, and the proximal portion of the stomach S.

Embodiments of satiation devices for controlling obesity are described herein that include a structure secured around the proximal portion of the stomach and below the gastro-esophageal junction Z. These devices are configured to create a small satiation pouch in the proximal portion of the stomach with a narrow passage into the lower portion of the stomach. The small satiation pouch is configured to collect a small amount of masticated food from the esophagus and the narrow passage delays emptying of the food from the small satiation pouch into the larger part of the stomach, thereby causing a feeling of fullness.

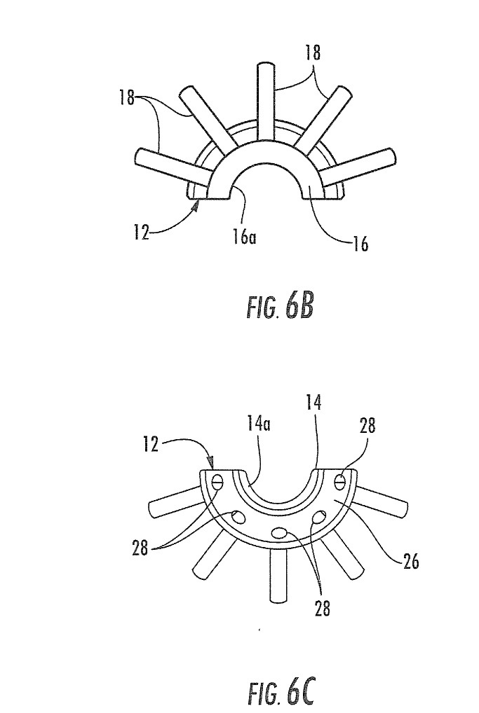

One embodiment of a satiation device 10 for controlling obesity is illustrated in FIGS. 2-3, 4A-4B, 5 and 6A-6C and includes a pair of mating sections 12. Each mating section 12 includes a proximal end portion 14 with an arcuate interior opening 14a, a distal end portion 16 with an arcuate interior opening 16a, and a plurality of elongated members 18 that extend between the proximal end portion 14 and distal end portion 16. The pair of mating sections 12 are configured to surround a portion of a stomach S below the gastro-esophageal junction thereof (and preferably below the z-line) and to be secured together in abutting relationship (FIG. 2). When secured together, the mating sections 12 form a chamber 20 with proximal and distal orifices 22, 24. The stomach portion contained within the chamber 20 forms a satiation pouch SP therein to receive food ingested by a patient. The distal orifice 24 forms a restrictive passage in the stomach that slows the passage of food from within the pouch SP to the lower portion of the stomach S.

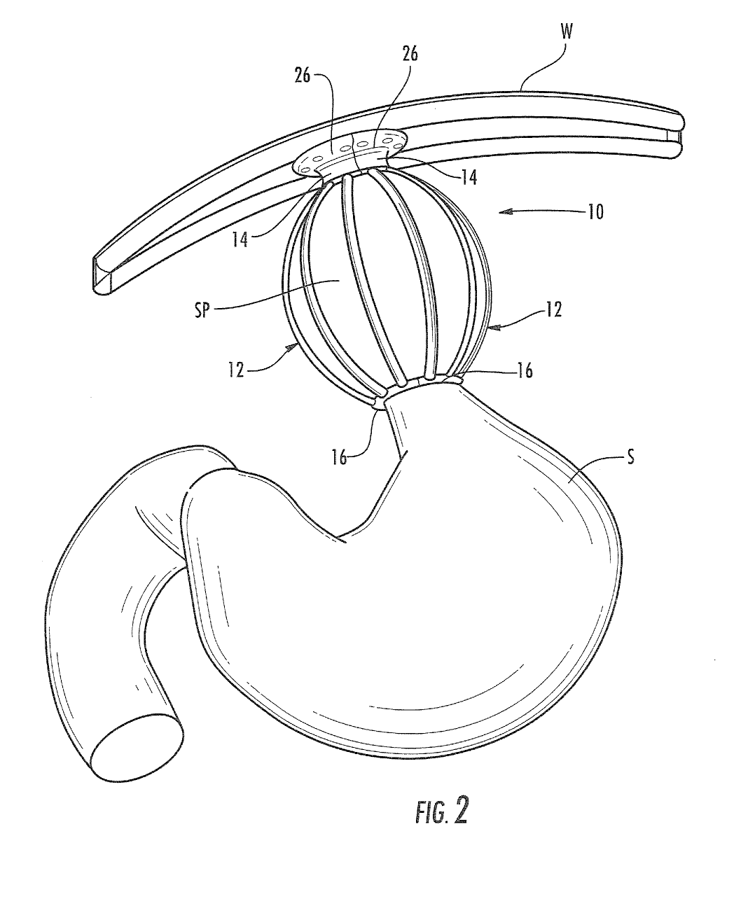

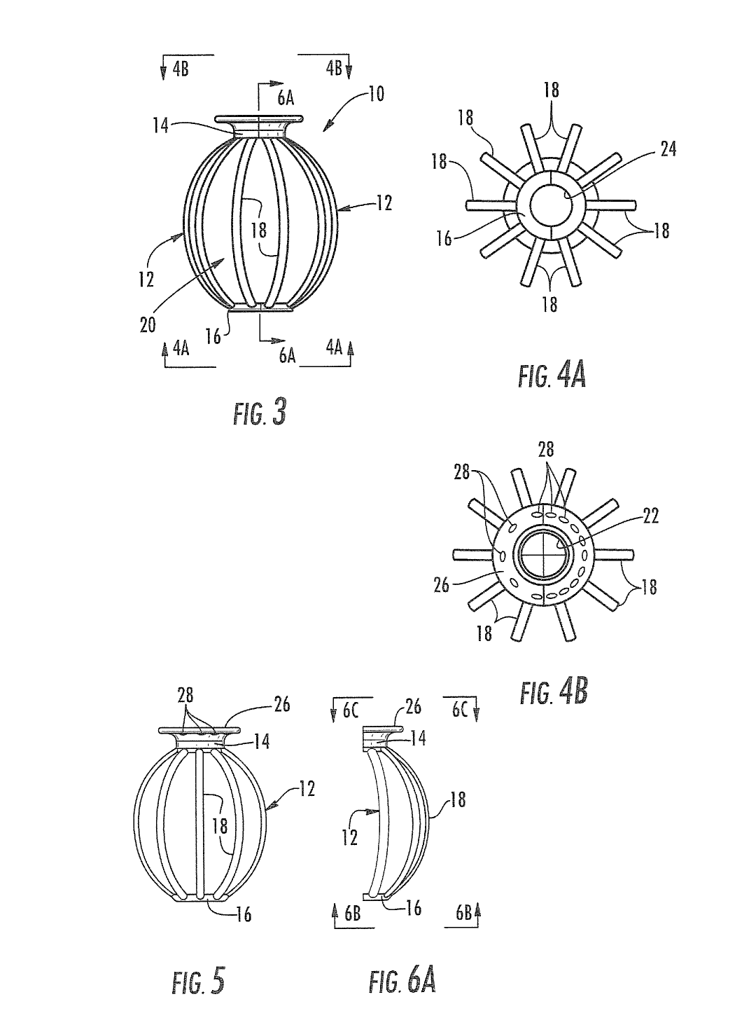

FIG. 3 is a side view of the satiation device 10 illustrating the pair of mating sections 12 secured together in abutting relationship. The illustrated satiation device 10 has a proximal orifice 22 with a generally circular configuration (FIG. 4B) and a distal orifice 24 with a generally circular configuration (FIG. 4A). However, the proximal and distal orifices 22, 24 may have various configurations and are not limited to generally circular configurations. For example, the proximal and distal orifices 22, 24 may have elongated, oval configurations, among others. Moreover, the proximal and distal orifices 22, 24 may have cross-sectional configurations selected for the particular stomach of a patient.

The proximal orifice 22 may be configured so as to not restrict the passage of food through the esophagus and into the satiation pouch SP retained within chamber 20. However, in some embodiments, it may be desirable to restrict the passage of food through the esophagus and into the pouch. As such, the proximal orifice 22 may have a configuration that restricts the stomach somewhat. The distal orifice 24 has a restrictive configuration so as to cause a narrow passage in a portion of the stomach between the satiation pouch SP and the lower portion of the stomach. This restrictive configuration slows the passage rate of food from the pouch SP into the lower portion of the stomach. For example, the distal orifice 24 may have a cross-sectional area of between about 0.05 square centimeters (0.05 cm²) and about 20 square centimeters (20 cm²).

In some embodiments, the size of the distal orifice 24 may be increased or decreased, for example by removing or installing spacers or inserts within the arcuate interior openings 16a of the mating sections 12, or by selecting mating sections 12 with different distal arcuate interior openings 16a and/or different distal end configurations. Alternatively, the size of the distal orifice 24 may be adjustable remotely (i.e., transabdominally) after implantation within a patient. For example, the size of the distal orifice 24 may be adjusted mechanically, magnetically, pneumatically, hydraulically, telemetrically using RF energy or ultrasound, among others. The adjustability of the distal orifice 24 enables a physician to set the distal orifice 24 to a size appropriate for a patient.

In some cases, it will also allow the physician to make adjustments to the distal orifice 24 after it has been implanted. For example, if the patient is not losing weight at a desired rate, the physician might reduce the size of the distal orifice 24 so that food will empty more slowly from the satiation pouch SP into the stomach. The physician might alternatively increase the size of the distal orifice 24 if necessary if weight loss is occurring too rapidly.

In the illustrated embodiment, the proximal end portion 14 of each mating section 12 includes a flange portion 26 that is configured to be attached to a body cavity wall W, for example the diaphragm of a patient. Flange portions 26 may have various configurations to facilitate attachment of the satiation device 10 to a body cavity wall and need not have the illustrated configuration. The illustrated flange portions 26 include a plurality of spaced-apart apertures 28 which are provided to facilitate attachment of the device 10 to a body cavity wall W via fasteners inserted therethrough and through or in a body cavity wall W. However, the apertures 28 are not required. In some embodiments, the material of the flange portions 26 may be selected such that fasteners can be inserted therethrough without the need for apertures. Exemplary fasteners for attaching the device 10 to a body cavity wall include, but are not limited to, sutures, clips, t-bar connectors, etc. In addition, the device 10 may be attached to a body cavity wall W via bio-compatible adhesives.

The flange apertures 28 may have various configurations and numbers without limitation. Although each flange portion 26 is illustrated as having about ten apertures 28, more apertures or fewer apertures may be utilized. Moreover, the flange portions 26 may be reinforced in the locations of the apertures 28. Each flange portion 26 and/or area surrounding an aperture 28 may be made of a suitably dense radio-opaque material, such as titanium, gold, or barium, to add in visualization of the device 10 during or after the installation within a patient. Each flange portion 26 and/or area surrounding an aperture 28 may also be marked using a different color to facilitate identification and orientation of fasteners.

Although not required, In some embodiments of the present invention, one or both of the distal end portions 16 may be secured to the stomach. Attachment may include, but is not limited to, the use of bio-compatible adhesive, fasteners, etc.

In some embodiments of the present invention, the mating sections 12 may be secured together via bio-compatible adhesive in the proximal and/or distal end portions. In other embodiments, the mating sections 12 may be secured together via one or more snap connectors in the proximal end portion and/or in the distal end portion. In other embodiments, the mating sections 12 may be secured together via one or more fasteners (e.g., screws, bolts, rivets, clips, t-bar connectors, sutures, etc.).

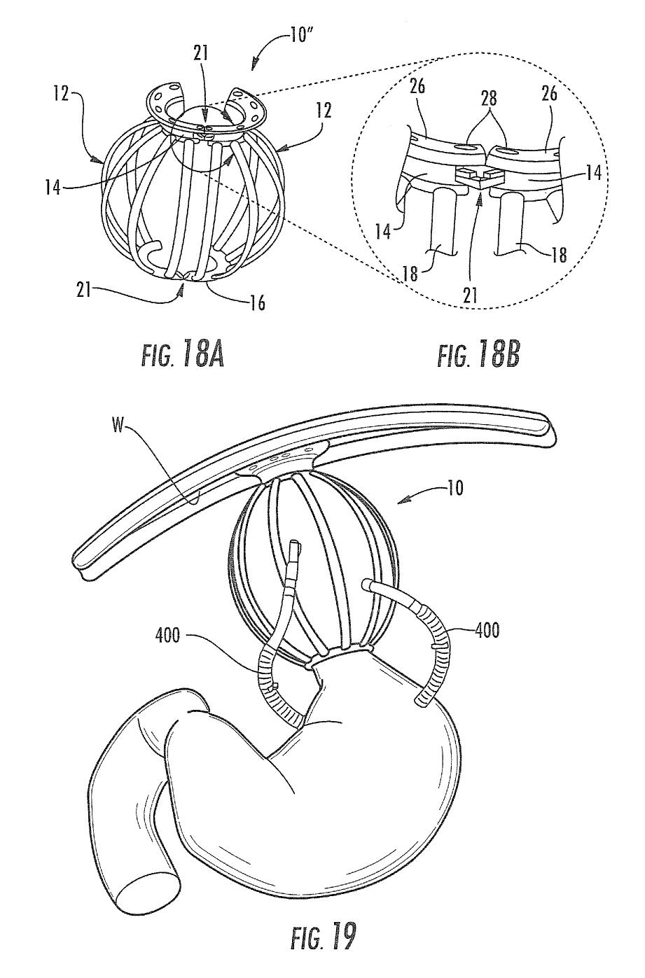

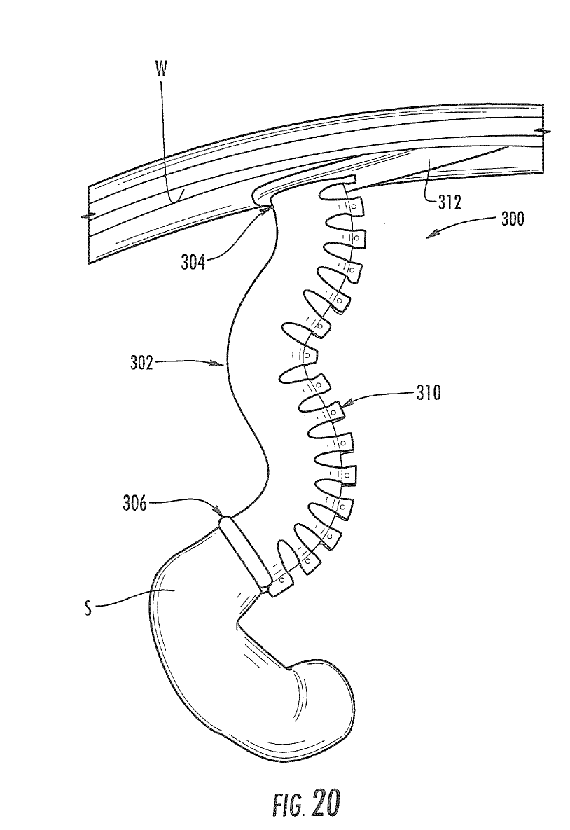

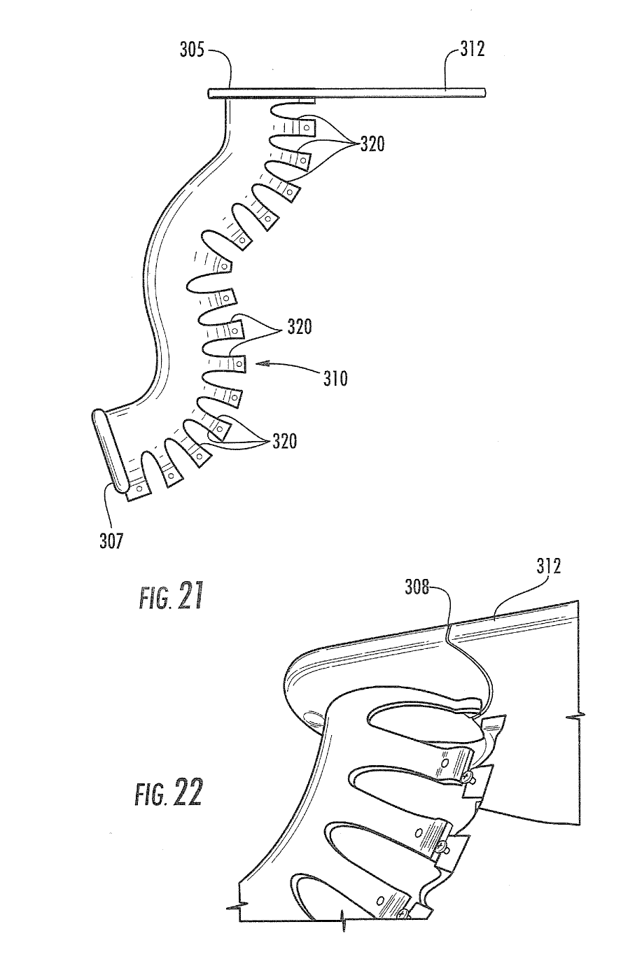

In some embodiments, the two mating sections 12 may be hingedly connected, rather than being separate components, so as to be movable relative to each other between open and joined configurations. For example, the two mating sections 12 may be hingedly connected at respective proximal end portions 14 and/or distal end portions 16 and/or at respective adjacent elongated members 18. Such a configuration may facilitate implantation within a patient as a single component, rather than as individual components. Moreover, a hinge connection between the two mating sections 12 may facilitate accurate alignment of the two mating sections 12 when joined together around a portion of the stomach of a patient. FIGS. 17A-17B illustrate the mating sections 12 of a satiation device 10′ connected together via a hinge 19 along respective adjacent elongated members 18. FIGS. 18A-18B illustrate the mating sections 12 of a satiation device 10″ connected together via a hinge 21 at respective proximal and distal end portions 14, 16.

In some embodiments, the elongated members 18 are flexible, rope-like elements. When food is ingested by a patient, the pouch SP expands outwardly against the flexible members 18 until the flexible members 18 become taut, thereby defining a predetermined pouch volume. In other embodiments, the elongated members 18 have an arcuate, rigid configuration. When food is ingested by a patient, the pouch SP expands outwardly until restrained by the rigid members 18, thereby defining a predetermined pouch volume. In either embodiment, the elongated members 18 form a chamber that restricts the volume of the pouch to a predetermined volume. For example, the pouch may be limited to a volume of less than about 300 cc. In some embodiments, the pouch SP is limited to a volume of between about 10 cc and about 30 cc, and may be as small as 2 cc. Because of its relatively small volume, the pouch SP functions to limit the amount of food that can be consumed at one time.

In the illustrated embodiment, the elongated members 18 define a generally globe-shaped chamber 20 when the pouch SP is expanded with food ingested by a patient. When the elongated members 18 are rigid, arcuate members, the chamber 20 maintains its globe-shaped configuration, even when the pouch SP does not contain food. When the elongated members 18 are flexible, rope-like members, the chamber 20 may not have a globe-shaped configuration when the pouch SP does not contain food. When food is ingested and the pouch expands outwardly, the elongated members 18 become taut in a globe-shaped configuration. However, satiation devices, according to embodiments of the present invention, are not limited to chambers with globe-shaped configurations. Chambers with various configurations may be utilized.

The illustrated mating sections 12 are substantially identical. However, embodiments of the present invention are not limited to identical mating sections. A mating section 12 can have a different shape, size, and/or configuration from another mating section(s) 12, according to other embodiments of the present invention.

The illustrated mating sections 12 may be formed from various materials including, but not limited to polymeric materials, metals, fabric, mesh, and combinations thereof. Each mating section 12 may be entirely formed from the same material or different portions of each mating section 12 may be formed from different materials. In addition, one portion of each mating section 12 may be bioabsorbable and another portion of each mating section 12 may be permanent. Exemplary materials are described below.

According to some embodiments of the present invention, the illustrated satiation device 10 may be configured to be removed from a patient. For example, the mating sections 12 may be joined together such that they can be separated from each other at a later point in time (i.e., the mating sections 12 are removably secured). In other embodiments of the present invention, the illustrated satiation device 10 may be designed to be permanently implanted within a patient.

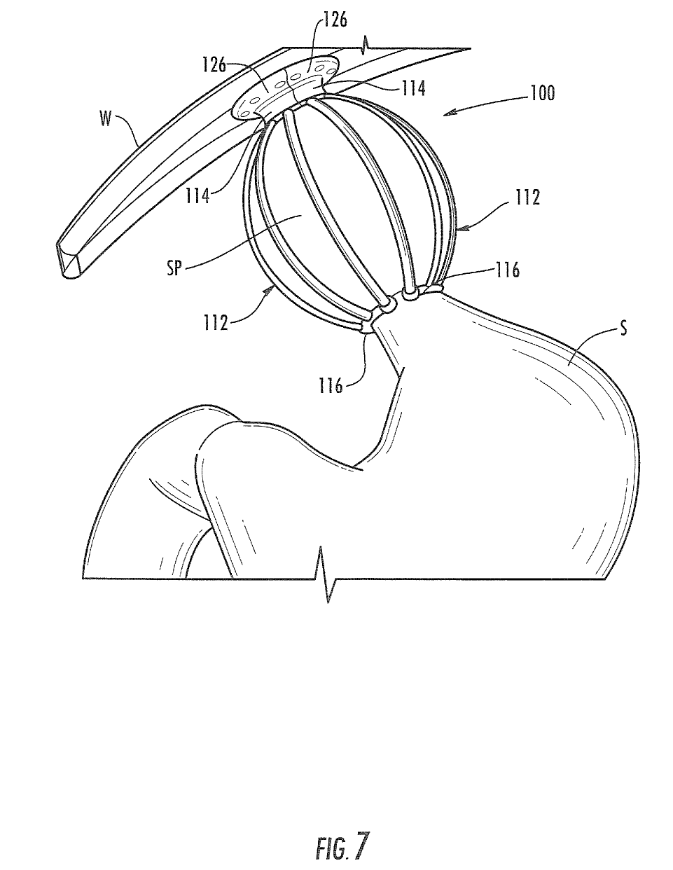

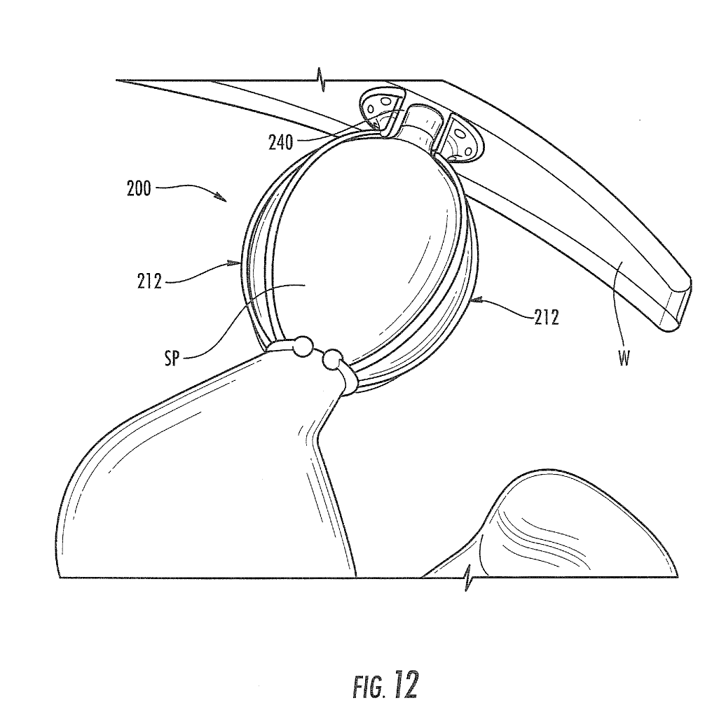

A satiation device 100 for controlling obesity, according to other embodiments of the present invention, is illustrated in FIGS. 7-8, 9A-9B, 10 and 11A-11B. The device 100 includes a pair of mating sections 112, but differs from the embodiment of FIGS. 2-3, 4A-4B, 5 and 6A-6C in that the distal end portions of the mating sections 112 do not extend completely around the circumference of the stomach S, as illustrated.

Each illustrated mating section 112 includes a proximal end portion 114 with an arcuate interior opening 114a, a distal end portion 116 with an arcuate interior opening 116a, and a plurality of elongated members 118 that extend between the proximal end portion 114 and distal end portion 116. The pair of mating sections 112 are configured to surround a portion of a stomach S below the gastro-esophageal junction thereof (and preferably below the z-line) and to be secured together in abutting relationship (FIG. 7). When secured together, the mating sections 112 form a chamber 120 with proximal and distal orifices 122, 124. The stomach portion contained within the chamber 120 forms a satiation pouch SP therein to receive food ingested by a patient and the distal orifice 124 forms a restrictive passage in the stomach that slows the passage of food from within the pouch SP to the lower portion of the stomach S, as described above.

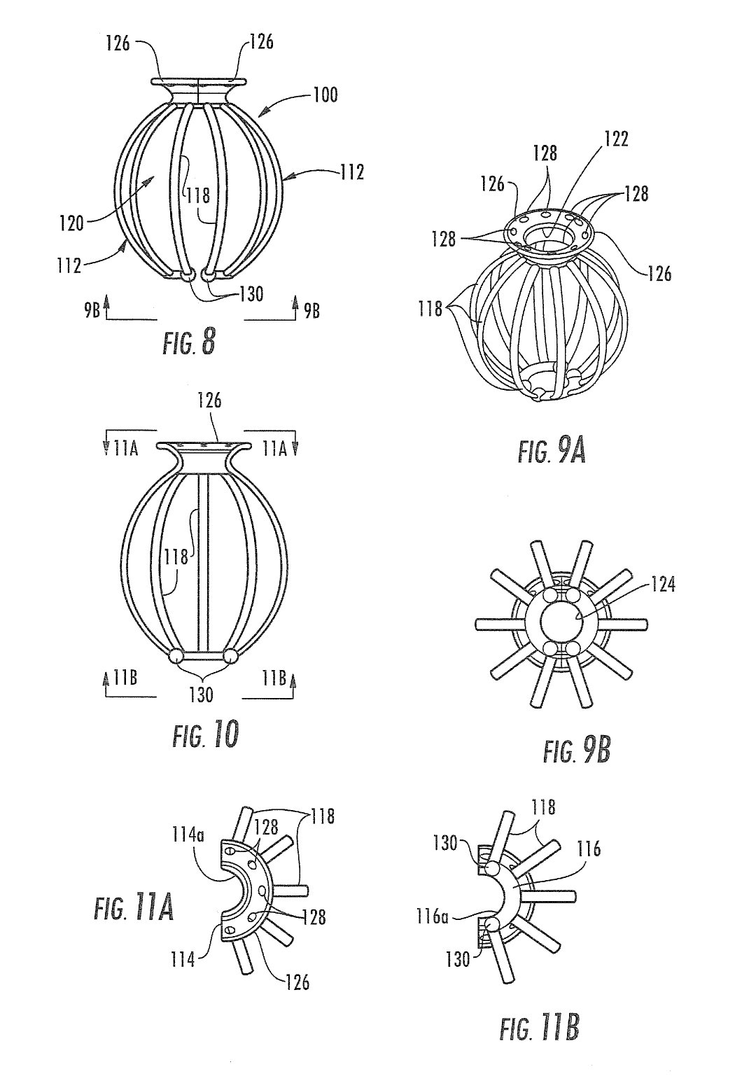

FIG. 8 is a side view of the satiation device 100 illustrating the pair of mating sections 112 secured together in abutting relationship. The illustrated satiation device 100 has a proximal orifice 122 (FIG. 9A) and a distal orifice 124 (FIG. 9B). The proximal and distal orifices 122, 124 may have various configurations and are not limited to the illustrated configurations. For example, the proximal and distal orifices 122, 124 may have elongated, oval configurations, among others. Moreover, the proximal and distal orifices 122, 124 may have cross-sectional configurations selected for the particular stomach of a patient.

The proximal orifice 122 may be configured so as to not restrict the passage of food through the esophagus and into the pouch SP retained within chamber 120. However, as described above, in some embodiments, it may be desirable to restrict the passage of food through the esophagus and into the pouch SP. As such, the proximal orifice 122 may have a configuration that restricts the stomach somewhat. The distal orifice 124 has a restrictive configuration so as to cause a narrow passage in a portion of the stomach, as described above. In some embodiments, the size of the distal orifice 124 may be adjustable before and/or after implantation within a patient, as described above. For example, the distal orifice 124 may have a cross-sectional area of between about 0.05 square centimeters (0.05 cm²) and about 20 square centimeters (20 cm²).

In the illustrated embodiment, the proximal end portion 114 of each mating section 112 includes a flange portion 126 that is configured to be attached to a body cavity wall W, for example the diaphragm of a patient. As described above, flange portions 126 may have various configurations to facilitate attachment of the satiation device 100 to a body cavity wall and need not have the illustrated configuration. The illustrated flange portions 126 include a plurality of spaced-apart apertures 128 which are provided to facilitate attachment of the device 100 to a body cavity wall W via fasteners inserted therethrough and through or in a body cavity wall W. However, the apertures 128 are not required. In some embodiments, the material of the flange portions 126 may be selected such that fasteners can be inserted therethrough without the need for apertures. The flange portions 126 may be attached to a body cavity wall in various ways, as described above.

In the illustrated embodiment, the distal end portion 116 of each mating section 112 includes an enlarged portion 130 at each free end thereof. Enlarged portions 130 are configured to have a smooth surface so as not to cause damage to the stomach, which otherwise may occur with a free end having edges.

Although not required, In some embodiments of the present invention, the one or both of the distal end portions 116 may be secured to the stomach. Attachment may include, but is not limited to, the use of bio-compatible adhesive, fasteners, etc.

The mating sections 112 may be secured together at the proximal end portions 114 via bio-compatible adhesive. In other embodiments, the mating sections 112 may be secured together at the proximal end portions 114 via one or more snap connectors. In other embodiments, the mating sections 112 may be secured together at the proximal end portions 114 via one or more fasteners (e.g., screws, bolts, rivets, clips, t-bar connectors, sutures, etc.).

In some embodiments, the two mating sections 112 may be hingedly connected, rather than being separate components, so as to be movable relative to each other between open and joined configurations. For example, the two mating sections 112 may be hingedly connected at respective proximal end portions 114 and/or at respective adjacent elongated members 118. Such a configuration may facilitate implantation within a patient as a single component, rather than as individual components. Moreover, a hinge connection between the two mating sections 112 may facilitate accurate alignment of the two mating sections 112 when joined together around a portion of the stomach of a patient.

As described above, the elongated members 118 may be flexible, rope-like elements or arcuate rigid elements. In either embodiment, the elongated members 118 form a chamber that restricts the volume of the pouch SP to a predetermined volume (e.g., between 2 cc and 300 cc; between 10 cc and 30 cc, etc.). In the illustrated embodiment, the elongated members 118 define a generally globe-shaped chamber 120 when the pouch is expanded with food ingested by a patient. However, chambers with various configurations may be utilized.

The illustrated mating sections 112 are substantially identical. However, embodiments of the present invention are not limited to identical mating sections. A mating section 112 can have a different shape, size, and/or configuration from another mating section(s) 112, according to other embodiments of the present invention.

The illustrated mating sections 112 may be formed from various materials including, but not limited to polymeric materials, metals, fabric, mesh, and combinations thereof. Each mating section 112 may be entirely formed from the same material or different portions of each mating section 112 may be formed from different materials. In addition, one portion of each mating section 112 may be bioabsorbable and another portion of each mating section 112 may be permanent. Exemplary materials are described below.

According to some embodiments of the present invention, the illustrated satiation device 100 may be configured to be removed from a patient.

For example, the mating sections 112 may be joined together such that they can be separated from each other at a later point in time (i.e., the mating sections 112 are removably secured). In other embodiments of the present invention, the illustrated satiation device 100 may be designed to be permanently implanted within a patient.

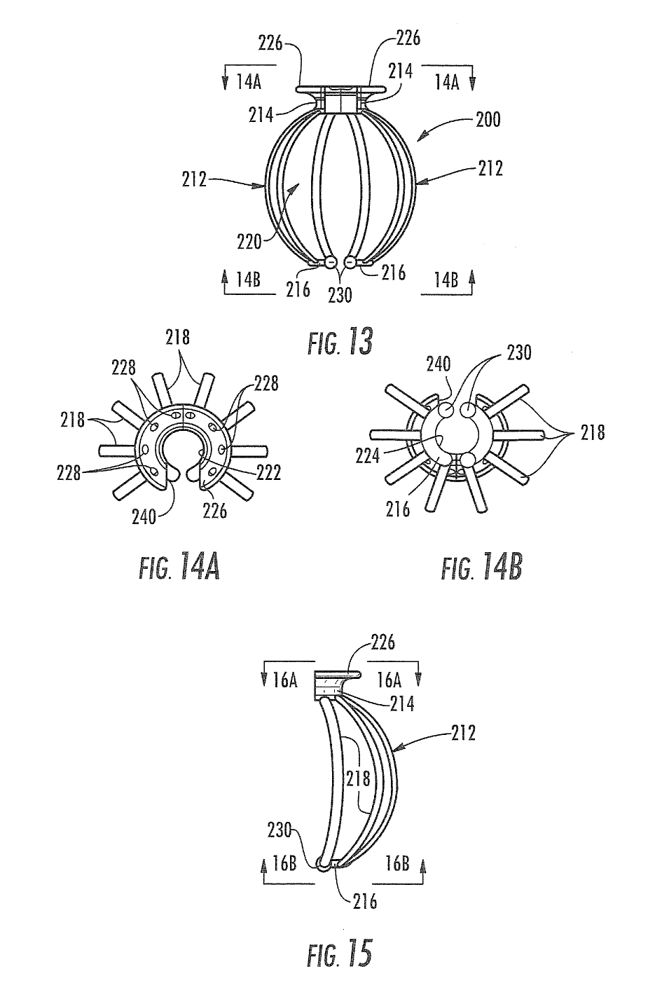

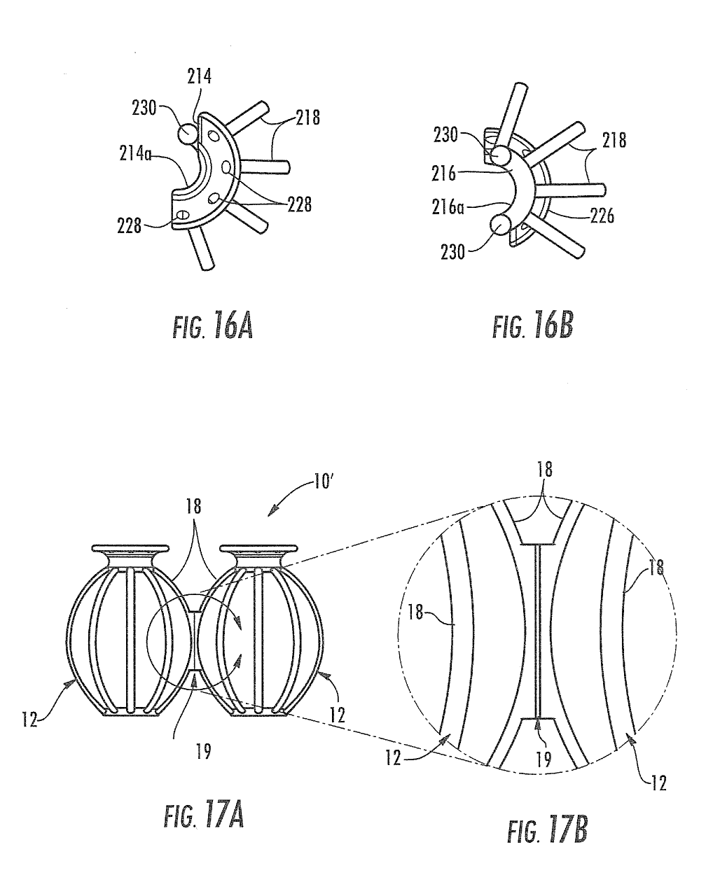

A satiation device 200 for controlling obesity, according to other embodiments of the present invention, is illustrated in FIGS. 12-13, 14A-14B, 15 and 16A-16B. The device 200 includes a pair of mirror-image mating sections 212, and is similar to the embodiment of FIGS. 7-8, 9A-9B, 10 and 11A-11B in that the distal end portions of the mating sections 212 do not extend completely around the circumference of the stomach S. However, this embodiment differs from the embodiment of FIGS. 7-8, 9A-9B, 10 and 11A-11B in that the proximal end portions 214 do not extend completely around the circumference of the stomach S, as illustrated.

Each illustrated mating section 212 includes a proximal end portion 214 with an arcuate interior opening 214a, a distal end portion 216 with an arcuate interior opening 216a, and a plurality of elongated members 218 that extend between the proximal end portion 214 and distal end portion 216. The pair of mating sections 212 are configured to surround a portion of a stomach S below the gastro-esophageal junction thereof (and preferably below the z-line) and to be secured together in abutting relationship (FIG. 12). When secured together, the mating sections 212 form a chamber 220 with proximal and distal orifices 222, 224. The stomach portion contained within the chamber 220 forms a satiation pouch SP therein to receive food ingested by a patient and the distal orifice 224 forms a restrictive passage in the stomach that slows the passage of food from within the pouch to the lower portion of the stomach S, as described above.

FIG. 13 is a side view of the satiation device 200 illustrating the pair of mating sections 212 secured together in abutting relationship. The illustrated satiation device 200 has a proximal orifice 222 (FIG. 14A) and a distal orifice 224 (FIG. 14B). However, the proximal and distal orifices 222, 224 may have various configurations and are not limited to the illustrated configurations. For example, the proximal and distal orifices 222, 224 may have elongated, oval configurations, among others. Moreover, the proximal and distal orifices 222, 224 may have cross-sectional configurations selected for the particular stomach of a patient.

The proximal orifice 222 may be configured so as to not restrict the passage of food through the esophagus and into the pouch retained within chamber 220. However, as described above, in some embodiments, it may be desirable to restrict the passage of food through the esophagus and into the pouch. As such, the proximal orifice 222 may have a configuration that restricts the stomach somewhat. The distal orifice 224 has a restrictive configuration so as to cause a narrow passage in a portion of the stomach. This restrictive configuration slows the passage rate of food from the pouch SP into the lower portion of the stomach. In some embodiments, the size of the distal orifice 224 may be adjustable before and/or after implantation within a patient, as described above. For example, the distal orifice 224 may have a cross-sectional area of between about 0.05 square centimeters (0.05 cm²) and about 20 square centimeters (20 cm²).

In the illustrated embodiment, the proximal end portion 214 of each mating section 212 includes a flange portion 226 that is configured to be attached to a body cavity wall W, for example the diaphragm of a patient. As described above, flange portions 226 may have various configurations to facilitate attachment of the satiation device 200 to a body cavity wall and need not have the illustrated configuration. The illustrated flange portions 226 include a plurality of spaced-apart apertures 228 which are provided to facilitate attachment of the device 200 to a body cavity wall W via fasteners inserted therethrough and through or in a body cavity wall W. However, the apertures 228 are not required. In some embodiments, the material of the flange portions 226 may be selected such that fasteners can be inserted therethrough without the need for apertures. The flange portions 226 may be attached to a body cavity wall in various ways, as described above.

In the illustrated embodiment, the distal end portion 216 of each mating section 212 includes an enlarged portion 230 at each free end thereof. Enlarged portions 230 are configured to have a smooth surface so as not to cause damage to the stomach, which otherwise may occur with a free end having edges. Although not required, In some embodiments of the present invention, the one or both of the distal end portions 216 may be secured to the stomach. Attachment may include the use of bio-compatible adhesive, fasteners, etc.

The proximal end portion 214 of each mating section 212 is less than semicircular, as illustrated. As such, there is a gap 240 between the proximal end portions 214 when the mating sections 212 are joined together around a stomach portion (FIG. 12). This gap 240 may facilitate installation of the device 200 by making it easier to insert the proximal end portions 214 around the gastro-esophageal junction region.

The mating sections 212 are secured together along one side of the proximal end portions 214 as illustrated in FIG. 13 and FIG. 14A. In some embodiments, the proximal end portions 214 of the mating sections 212 are secured together via bio-compatible adhesive. In other embodiments, proximal end portions 214 of the mating sections 212 may be secured together via one or more snap connectors in the proximal end portions 214. In other embodiments, the proximal end portions 214 of the mating sections 212 may be secured together via one or more fasteners (e.g., screws, bolts, rivets, clips, t-bar connectors, sutures, etc.).

In some embodiments, the two mating sections 212 may be hingedly connected, rather than being separate components, so as to be movable relative to each other between open and joined configurations. For example, the two mating sections 212 may be hingedly connected at respective proximal end portions 214 and/or at respective adjacent elongated members 218. Such a configuration would facilitate insertion within a patient as a single component, rather than as individual components. Moreover, a hinge connection between the two mating sections 212 may facilitate accurate alignment of the two mating sections 112 when joined together around a portion of the stomach of a patient.

In some embodiments, the two mating sections 212 may be rigidly connected, rather than being separate components, prior to installation within a patient. For example, the two mating sections 212 may be connected at the proximal end portions 214 (or the device 200 may be a single unitary device, i.e., no separate components). Such a configuration may facilitate implantation within a patient. The gap 240, as described above, may facilitate accurate alignment of the device 200 installed around a portion of the stomach of a patient. In this embodiment, the distal end portions 216 may be capable of flexing outwardly to facilitate insertion of the device 200 around a stomach.

As described above, the elongated members 218 may be flexible, rope-like elements or arcuate rigid elements. In either embodiment, the elongated members 218 form a chamber that restricts the volume of the pouch SP to a predetermined volume (e.g., between 2 cc and 300 cc; between 10 cc and 30 cc, etc.). In the illustrated embodiment, the elongated members 218 define a generally globe-shaped chamber 220 when the pouch SP is expanded with food ingested by a patient. However, chambers with various configurations may be utilized.

The illustrated mating sections 212 may be formed from various materials including, but not limited to polymeric materials, metals, fabric, mesh, and combinations thereof. Each mating section 212 may be entirely formed from the same material or different portions of each mating section 212 may be formed from different materials. In addition, one portion of each mating section 212 may be bioabsorbable and another portion of each mating section 212 may be permanent. Exemplary materials are described below.

According to some embodiments of the present invention, the illustrated satiation device 200 may be configured to be removed from a patient. For example, the mating sections 212 may be joined together such that they can be separated from each other at a later point in time (i.e., the mating sections 212 are removably secured). In other embodiments of the present invention, the illustrated satiation device 200 may be designed to be permanently implanted within a patient.

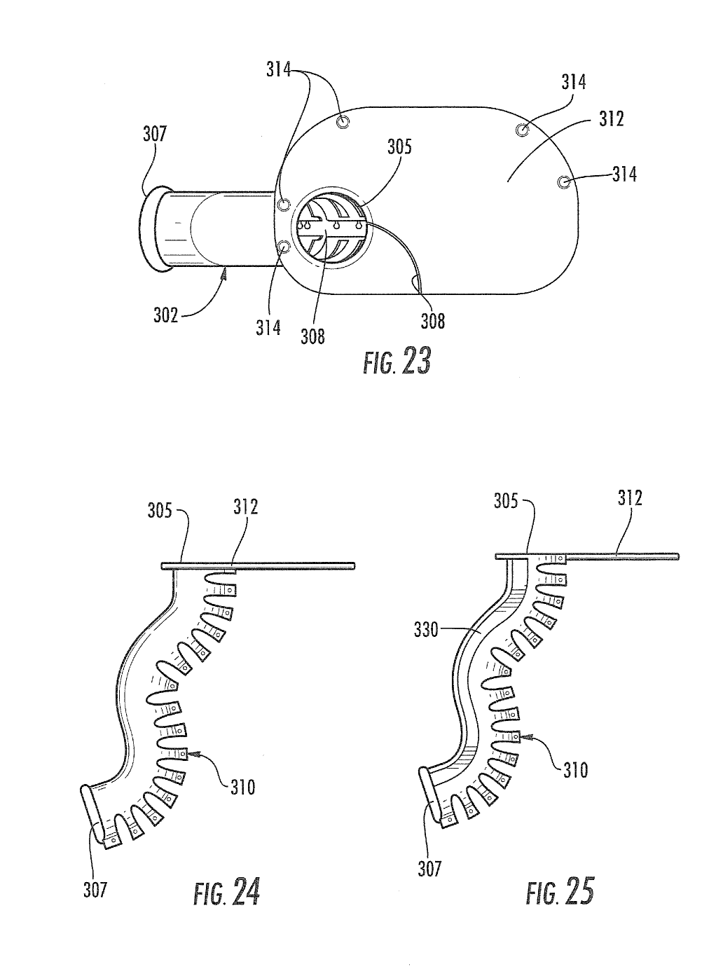

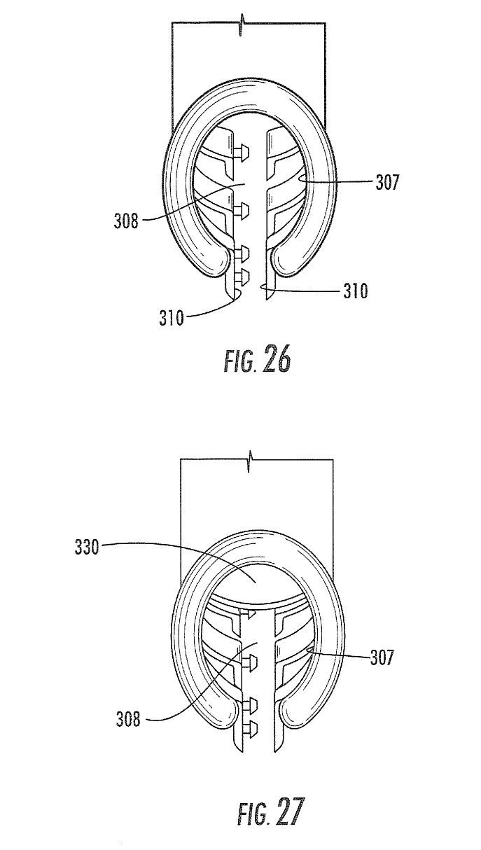

A satiation device 300 for controlling obesity, according to other embodiments of the present invention, is illustrated in FIGS. 20-29. The device 300 is configured to wrap around and restrain the stomach of a patient. The device 300 may extend, for example, from the LES to the pyloric sphincter (PS). However, embodiments of the device 300 may extend along any length of a stomach and are not limited to extending only from the LES to the PS.

The illustrated device 300 includes a flexible housing 302 with a proximal end portion 304 that defines a proximal orifice 305 and a distal end portion 306 that defines a distal orifice 307. The housing 302 includes a slit 308 along the length thereof that enables the housing 302 to be wrapped around a stomach S and extend from the respective proximal and distal orifices 305, 307, as illustrated. Opposing slit edges 310 are configured to be secured together when the housing 302 is wrapped around a stomach to secure the device 300 thereto. The stomach S restrained within the housing 302 is prevented from expanding when food is ingested thereinto, thereby quickly causing a feeling of fullness to a patient.

The housing proximal end portion 304 includes a flange 312 that is configured to be attached to a body cavity wall W, for example the diaphragm of a patient. The flange 312 may have various configurations to facilitate attachment of the satiation device 300 to a body cavity wall and need not have the illustrated configuration. The illustrated flange 312 includes a plurality of spaced-apart apertures 314 which are provided to facilitate attachment of the device 300 to a body cavity wall W via fasteners inserted therethrough and through or in a body cavity wall W. However, the apertures 314 are not required. In some embodiments, the material of the flange 312 may be selected such that fasteners can be inserted therethrough without the need for apertures. Exemplary fasteners for attaching the device 300 to a body cavity wall include, but are not limited to, sutures, clips, t-bar connectors, etc. In addition, the device 300 may be attached to a body cavity wall via bio-compatible adhesives.

The flange apertures 314 may have various configurations and numbers without limitation. Although the illustrated flange 312 has five apertures 314, more apertures or fewer apertures may be utilized. Moreover, the flange 312 may be reinforced in the locations of the apertures 314 (or at other locations), for example via insert molding techniques, etc. The flange 312 and/or other portions of the housing 302 may be made of a suitably dense radio-opaque material, such as titanium, gold, or barium to add in visualization of the device 300 during or after the installation within a patient. The flange 312 and/or portions of the housing 302 may also be marked using a different color to facilitate identification and orientation of fasteners, etc.

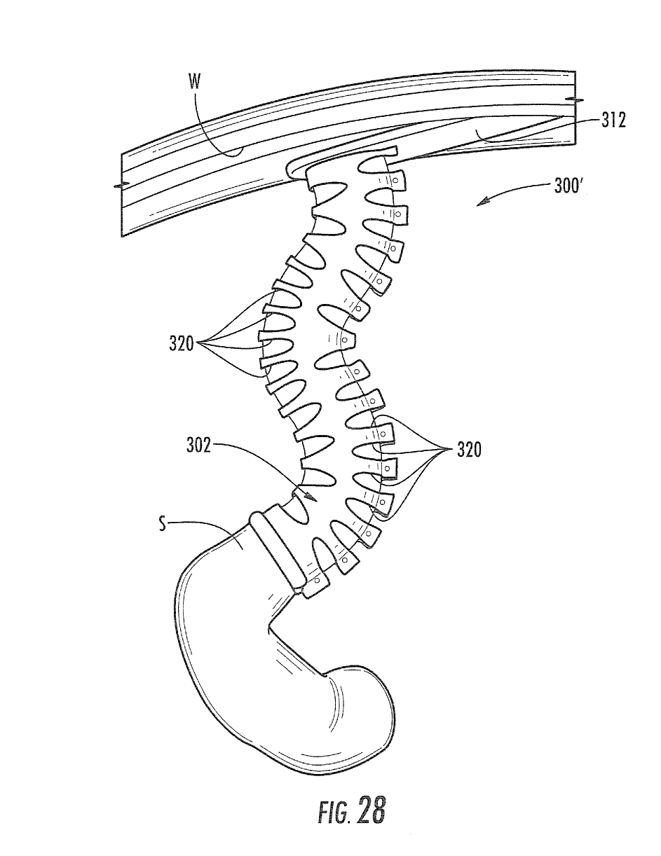

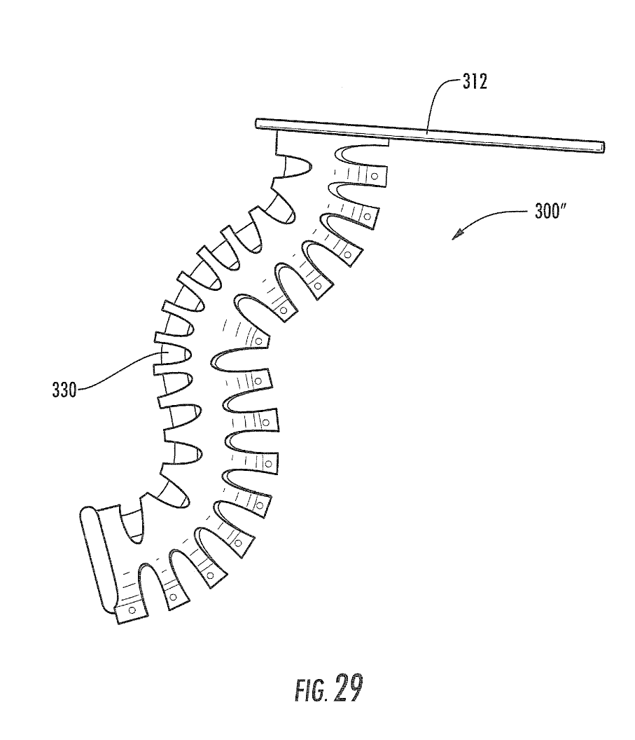

The slit edges 310 include openings 320, as illustrated, for arteries and veins that extend from a stomach. A satiation device 300′, according to other embodiments of the present invention, is illustrated in FIG. 28 and includes an additional set of openings 320 on the other side of the illustrated housing 302. Satiation devices according to embodiments of the present invention may have openings with virtually any shape, size, and location, and may have any number of openings, as well, without limitation. The openings 320 may also serve to add flexibility to the housing 302, thereby facilitating implantation. Although the openings 320 allow the passage of arteries and veins therethrough, they are small enough to prevent portions of the stomach from extending therethrough (referred to as “out pouching”) and the resulting stasis of food.

The slit edges 310 are configured to be joined together via fasteners (e.g., screws, bolts, rivets, clips, t-bar connectors, sutures, etc.), or via adhesive, as described above with respect to other embodiments of the present invention.

Although not required, In some embodiments of the present invention, the distal end portion 306 may be secured to the stomach. Attachment may include the use of bio-compatible adhesive, fasteners, etc.

In some embodiments, the size of the housing distal portion 306 may be increased or decreased, for example by removing or installing spacers or inserts within the housing distal orifice 307 and/or via an expandable and retractable element. Alternatively, the size of the distal orifice 307 may be adjustable remotely (i.e., transabdominally) after implantation within a patient. For example, the size of the distal orifice 307 may be adjusted mechanically, magnetically, pneumatically, hydraulically, and/or telemetrically via RF energy or ultrasound, among others. The adjustability of the distal orifice 307 enables a physician to set the distal orifice 307 to a size appropriate for a patient. In some cases, it will also allow the physician to make adjustments to the distal orifice 307 after the device 300 has been implanted.

Referring to FIG. 29, a satiation device 300″ may include an expandable liner or balloon 330 that facilitates proper sizing of the housing 302 around the stomach of a patient. The sizing balloon 330 may be expanded during implantation and/or may be remotely expanded and/or retracted after implantation, such as, for example, via an external fill port and fill tube. The sizing balloon may be expanded via a fluid or via a gas. In the illustrated embodiment, the sizing balloon 330 is positioned within the housing 302 along the side of lesser curvature. However, embodiments of the present invention are not limited to the illustrated position of the sizing balloon 330. The sizing balloon 330 may be positioned in various other configurations and locations within the housing 302. For example, the sizing balloon 330 may be positioned within the housing on the opposite side, referred to as the side of greater curvature.

### Materials

Satiation devices (10, 10′, 10″, 100, 200, 300, 300′, 300″), according to some embodiments of the present invention, may be formed from various materials including, but not limited to polymeric materials and metals, including polymeric meshes and fabrics and metallic meshes and fabrics. Exemplary fabrics may include woven fabrics, nonwoven fabrics, a knitted fabrics, braid fabrics, etc. Satiation devices (10, 10′, 10″, 100, 200, 300, 300′, 300″), according to embodiments of the present invention, may be formed from antiadhesive materials or non tissue adhering materials.

Satiation devices (10, 10′, 10″, 100, 200, 300, 300′, 300″), according to some embodiments of the present invention, may be formed from or coated with materials having various combinations of silicone and metal, including composites and hybrids and blends thereof.

Satiation devices (10, 10′, 10″, 100, 200, 300, 300′, 300″), according to some embodiments of the present invention, may be formed in various ways including, but not limited to, molding, insert molding, casting, machining, etc.

In addition, satiation devices, according to some embodiments of the present invention, may be formed from or coated with polymeric material. For example, one or more portions of the mating sections (12, 112, 212) of the various embodiments may be formed from (or coated with) polymeric material. Exemplary polymeric materials that may be utilized include, but are not limited to, elastomers, rubbers (e.g., nitrile, latex, etc.), polyurethanes (e.g., ChronoFlex® polyurethane, etc.), polyolefins, poly(meth)acrylates, polyesters (e.g., Dacron®) polyester), ePTFE fabric (e.g., GoreTex® fabric or others), polyamides, polyvinyl resins, silicon resins, polycarbonates, polyfluorocarbon resins, synthetic resins, polystyrene, nylon fabrics, silicone, bio-absorbable materials (e.g., PLLA, PGA, PCL, poly-amhydride etc).

Moreover, satiation devices, according to some embodiments of the present invention, may be formed of a composite of compliant, semi-compliant and/or non-compliant materials which give different regions of the devices (10, 10′, 10″, 100, 200, 300, 300′, 300″) different degrees of compliance so as to allow/limit expansion of the device in various locations. For example, it may be desirable to provide a device (10, 10′, 10″, 100, 200, 300, 300′, 300″) with fairly elastic distal end portions (16, 116, 216) so as to prevent occlusion in the event a large piece of food is ingested into the satiation pouch SP, whereas the proximal end (14, 114, 214) of the device may be stiffer. Varying degrees of compliance may also be built into the devices (10, 10′, 10″, 100, 200, 300, 300′, 300″) by varying the cross-sectional thickness of the devices in different regions thereof. In some embodiments, the device material may be coated with a lubricious, bio-compatible, chemically inert material, such as paraleyne, to reduce friction with a stomach.

In some embodiments, polymeric material utilized is non-erodible (or the device has a non-erodible coating), although in other embodiments it may be desirable for the polymeric material to be erodible (or the device may have an erodible coating). Exemplary erodible materials include, but are not limited to, surgical gut, silk, cotton, poly(hydroxybutyrate), polycarbonate, polyacrylate, polyanhydride, poly(ortho esters), poly(phosphoesters), polyesters, polyamides, polyphosphazenes, poly(p-dioxane), poly(amino acid), polyglactin, erodable hydrogels, collagen, chitosan, poly(lactic acid), poly(L-lactic acid), poly(D,L-lactic acid), poly(glycolic acid), poly(D-lactic-co-glycolic acid), poly(L-lactic-co-glycolic acid), poly (D,L-lactic-co-glycolic acid), poly(Ε-caprolactone), poly(valerolactone), poly(hydroxy butyrate), poly(hydrovalerate), polydioxanone, poly(propylene fumarate), poly(ethyleneoxide)-poly(butylenetetraphthalate), poly(lactic acid-co-lysine), poly(lactic acid-co-trimethylene carbonate), poly(L-lactic acid) and poly(Ε-caprolactone) copolymers, and blends thereof.

Exemplary non-erodible materials include, but are not limited to, fluoropolymers, polyesters, PET, polyethylenes, polypropylenes, etc., and/or ceramics, such as hydroxyapetite.

Exemplary metallic materials include titanium and platinum, metal alloys, such as stainless steel, nickel-titanium, and cobalt-chromium, etc.

One or more portions of the mating sections (12, 112, 212) of the various satiation device embodiments may be formed from shape memory material, such as nitinol.

Satiation devices, according to some embodiments of the present invention, may include various pharmacological agents. In general, pharmacological agents suitable for inclusion in materials and/or coatings (and according to embodiments of the present invention) include, but are not limited to, drugs and other biologically active materials, and may be intended to perform a variety of functions, including, but not limited to: anti-infection treatment, anti-inflammatory treatment, and the prevention of smooth muscle cell growth, migration, proliferation within a vessel wall. Pharmacological agents may include antineoplastics, antimitotics, antiinflammatories, antiproliferatives, antibiotics, and antiallergic substances as well as combinations thereof. Examples of antineoplastics and/or antimitotics include paclitaxel (cytostatic and ant-inflammatory) and its analogs and all compounds in the TAXOL® (Bristol-Myers Squibb Co., Stamford, Conn.) family of pharmaceuticals, docetaxel (e.g., TAXOTERE® from Aventis S. A., Frankfurt, Germany) methotrexate, azathioprine, vincristine, vinblastine, fluorouracil, doxorubicin hydrochloride (e.g., ADRIAMYCIN® from Pharmacia & Upjohn, Peapack N.J.), and mitomycin (e.g., MUTAMYCIN® from Bristol-Myers Squibb Co., Stamford, Conn.). Examples of antiinflammatories include Sirolimus and its analogs (including but not limited to Everolimus and all compounds in the Limus family of pharmaceuticals), glucocorticoids such as dexamethasone, methylprednisolone, hydrocortisone and betamethasone and non-steroidal antiinflammatories such as aspirin, indomethacin and ibuprofen. Examples of cytostatic or antiproliferative agents or proliferation inhibitors include everolimus, actinomycin D, as well as derivatives and analogs thereof (manufactured by Sigma-Aldrich, Milwaukee, Wis.; or COSMEGEN® available from Merck & Co., Inc., Whitehouse Station, N.J.), angiopeptin, angiotensin converting enzyme inhibitors such as captopril (e.g., CAPOTEN® and CAPOZIDE® from Bristol-Myers Squibb Co., Stamford, Conn.), cilazapril or lisinopril (e.g., Prinivilo and PRINZIDE® from Merck & Co., Inc., Whitehouse Station, N.J.); calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name MEVACOR® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents that may be used include alphainterferon, anti-Tumor Necrosis Factor (TNF) α, genetically engineered epithelial cells, and dexamethasone.

### Implantation

Implantation of the various satiation device embodiments may be performed using various procedures including, but not limited to, surgery, laparoscopy, endoscopy, and Natural Orifice Translumenal Endoscopic Surgery (NOTES). In a laparoscopic procedure, surgeons use small incisions (e.g., ¼ to ½ inch) to enter the abdomen through cannulas (narrow tube-like instruments). The laparoscope, which is connected to a tiny video camera, is inserted through the small cannula. A picture is projected onto a monitor or screen giving the surgeon a magnified view of the stomach and other internal organs. Five to six small incisions and cannulas are placed for use of specialized instruments to implant the various satiation devices (10, 10′, 10″, 100, 200, 300, 300′, 300″), according to embodiments of the present invention. The entire operation is performed inside the abdomen after expanding the abdomen with carbon dioxide (CO₂) gas. The CO₂ gas is removed at the completion of the operation. The abdominal space may also be expanded using other techniques including “gasless” laparoscopy.

In an endoscopic procedure, an endoscope is gently passed through the mouth, down the esophagus, and into the stomach and duodenum. The endoscope is connected to a tiny video camera from which a picture is projected onto a TV giving the surgeon a magnified view of the stomach and other internal organs. Specialized instruments to implant the various satiation devices (10, 10′, 10″, 100, 200, 300, 300′, 300″), according to embodiments of the present invention, are passed through the endoscope.

A NOTES procedure is a new type of surgical procedure currently being studied at research hospitals and facilities around the world. NOTES procedures have been developed because: patient recovery time can be reduced because the procedures are less invasive than conventional surgical procedures, because patients experience less physical discomfort than with traditional procedures, and because patients have virtually no visible scarring following this type of surgery. According to embodiments of the present invention, a doctor inserts a tube down the esophagus, makes a small incision in the stomach or digestive tract to gain access to the abdominal cavity and implants the various satiation devices (10, 10′, 10″, 100, 200, 300, 300′, 300″) of the present invention. FIG. 19 illustrates the implantation of the satiation device 10 of FIG. 2 around a portion of a stomach via a NOTES device inserted down the esophagus of a patient, and wherein arms or portions 400 of the NOTES device are extending through the stomach wall.

In the drawings and specification, there have been disclosed typical preferred embodiments of the invention and, although specific terms are employed, they are used in a generic and descriptive sense only and not for purposes of limitation, the scope of the invention being set forth in the following claims.

That which is claimed:

1. A satiation device configured to be positioned around a portion of a stomach of a patient so as to create a satiation pouch, the device comprising a plurality of mating sections, each mating section including:
a proximal end portion with an arcuate interior opening;
a distal end portion with an arcuate interior opening; and
a plurality of elongated members extending from the proximal end portion to the distal end portion;
wherein the plurality of mating sections are configured to surround a portion of a stomach and to be secured together to form a chamber with proximal and distal orifices, wherein the stomach portion within the chamber forms a satiation pouch therein to receive food ingested by the patient.

2. The device of claim 1, wherein two of the mating sections are hingedly connected at respective proximal end portions, at respective distal end portions, and/or at respective adjacent elongated members.

3. The device of claim 1, wherein the plurality of mating sections comprise a pair of mating halves.

4. The device of claim 3, wherein the mating halves are hingedly connected at respective proximal end portions, at respective distal end portions, and/or at respective adjacent elongated members.

5. The device of claim 1, wherein the chamber has a generally globe-shaped configuration.

6. The device of claim 1, wherein the chamber is configured such that the satiation pouch has a volume of less than about 300 cubic centimeters (cc).

7. The device of claim 1, wherein the chamber is configured such that the satiation pouch has a volume of between about 10 cc and about 30 cc.

8. The device of claim 1, wherein the proximal orifice has a generally circular configuration.

9. The device of claim 1, wherein the distal orifice has a generally circular configuration.

10. The device of claim 1, wherein the distal orifice has a restrictive configuration so as to cause a narrow passage in a portion of the stomach.

11. The device of claim 1, wherein a size of the distal orifice is adjustable.

12. The device of claim 11, wherein the size of the distal orifice is transabdominally adjustable mechanically, magnetically, pneumatically, hydraulically, and/or telemetrically via RF energy or ultrasound.

13. The device of claim 1, wherein the distal orifice has a cross-sectional area of between about 0.05 square centimeters (0.05 cm²) and about 20 square centimeters (20 cm²).

14. The device of claim 1, wherein the proximal end portion of each mating section comprises a flange portion configured to be attached to a body cavity wall.

15. The device of claim 14, wherein each flange portion comprises a plurality of spaced-apart apertures.

16. The device of claim 1, wherein the elongated members have a flexible configuration.

17. The device of claim 1, wherein the elongated members have an arcuate, rigid configuration.

18. The device of claim 1, wherein the mating sections comprise polymeric material.

19. The device of claim 18, wherein the polymeric material is erodible.

20. The device of claim 18, wherein the polymeric material is non-erodible.

21. The device of claim 1, wherein the mating sections comprise drug eluting material.

22. The device of claim 1, wherein one or more of the mating sections comprises one or more pharmacological agents.

23. The device of claim 1, wherein the mating sections comprise material configured to stimulate tissue growth.

24. The device of claim 1, wherein the mating sections comprise shape memory material.

25. The device of claim 24, wherein the shape memory material comprises nitinol.

26. The device of claim 24, wherein the shape memory material comprises a shape memory polymer.

27. The device of claim 1, wherein the mating sections comprise a mesh.

28. The device of claim 1, wherein the mating sections comprise metal.

29. The device of claim 1, wherein the mating sections are secured together via adhesive.

30. The device of claim 1, wherein the mating sections are secured together via one or more snap connectors in the proximal end portion, and/or via one or more snap connectors in the distal end portion.

31. The device of claim 1, wherein the mating sections are secured together via one or more fasteners.

32. The device of claim 1, wherein the mating sections are removably secured together.

33. A method of forming a satiation pouch in a portion of a stomach of a patient, comprising:
securing a plurality of mating sections together around a portion of the stomach, wherein each mating section includes:
a proximal end portion with an arcuate interior opening;
a distal end portion with an arcuate interior opening; and
a plurality of elongated members extending from the proximal end portion to the distal end portion, wherein the mating sections form a chamber with proximal and distal orifices, and wherein the stomach portion within the chamber forms a satiation pouch; and
attaching one or more of the proximal end portions to a body cavity wall of the patient.

34. The method of claim 33, wherein the plurality of mating sections are secured together such that the proximal end portions of the mating sections are positioned below the gastro-esophageal junction.

35. The method of claim 33, wherein the plurality of mating sections are secured together via a laparoscopy tool, via an endoscope, and/or via a NOTES device inserted down the esophagus of a patient.

36. The method of claim 33, further comprising adjusting the size of the distal orifice to a cross-sectional area of between about 0.05 square centimeters (0.05 cm²) and about 20 square centimeters (20 cm²).

37. The method of claim 33, wherein the one or more proximal end portions are attached to the body cavity wall via one or more surgical clips, one or more sutures, one or more staples, or via adhesive.

38. The method of claim 33, wherein the mating sections are secured together via one or more snap connectors in the proximal end portions, and/or via one or more snap connectors in the distal end portions.

39. The method of claim 33, wherein the mating sections are secured together via one or more fasteners.

40. The method of claim 33, wherein two of the mating sections are hingedly connected at respective proximal end portions, at respective distal end portions, and/or at respective adjacent elongated members.

41. The method of claim 33, wherein the plurality of mating sections comprise a pair of mating halves.

42. The method of claim 41, wherein the mating halves are hingedly connected at respective proximal end portions, at respective distal end portions, and/or at respective adjacent elongated members.

43. The method of claim 33, wherein securing the mating sections together comprises removably securing the mating sections together.

44. A satiation device configured to be positioned around a portion of a stomach of a patient so as to create a satiation pouch, the device comprising:
a proximal end portion with an arcuate interior opening;
a distal end portion with an arcuate interior opening; and
a plurality of elongated members extending from the proximal end portion to the distal end portion;
wherein the proximal and distal end portions are configured to partially surround the stomach to form a chamber with proximal and distal orifices, wherein the stomach portion within the chamber forms a satiation pouch therein to receive food ingested by the patient.

45. The device of claim 44, wherein the chamber has a generally globe-shaped configuration.

46. The device of claim 44, wherein the chamber is configured such that the satiation pouch has a volume of less than about 300 cubic centimeters (cc).

47. The device of claim 44, wherein the chamber is configured such that the satiation pouch has a volume of between about 10 cc and about 30 cc.

48. The device of claim 44, wherein the distal orifice has a restrictive configuration so as to cause a narrow passage in a portion of the stomach.

49. The device of claim 44, wherein a size of the distal orifice is adjustable.

50. The device of claim 49, wherein the size of the distal orifice is transabdominally adjustable mechanically, magnetically, pneumatically, hydraulically, and/or telemetrically via RF energy or ultrasound.

51. The device of claim 44, wherein the distal orifice has a cross-sectional area of between about 0.05 square centimeters (0.05 cm²) and about 20 square centimeters (20 cm²).

52. The device of claim 44, wherein the proximal end portion comprises a flange portion configured to be attached to a body cavity wall.

53. The device of claim 44, wherein the elongated members have a flexible configuration.

54. The device of claim 44, wherein the elongated members have an arcuate, rigid configuration.

55. The device of claim 44, wherein the device contains one or more pharmacological agents.

56. A method of forming a satiation pouch in a portion of a stomach of a patient, comprising:
implanting a unitary device around a portion of the stomach, wherein the device includes:
a proximal end portion with an arcuate interior opening;
a distal end portion with an arcuate interior opening; and
a plurality of elongated members extending from the proximal end portion to the distal end portion that form a chamber with proximal and distal orifices, and wherein the stomach portion within the chamber forms a satiation pouch; and
attaching one or more of the proximal end portions to a body cavity wall of the patient.

57. The method of claim 56, wherein the device is positioned below the gastro-esophageal junction.

58. The method of claim 56, wherein the device is removably secured around a portion of the stomach.

59. The method of claim 56, wherein the device is implanted via a laparoscopy tool, via an endoscope, and/or via a NOTES device inserted down the esophagus of a patient.

60. The method of claim 56, further comprising adjusting the size of the distal orifice to a cross-sectional area of between about 0.05 square centimeters (0.05 cm²) and about 20 square centimeters (20 cm²).

61. A satiation device, comprising a flexible housing configured to wrap around and restrain a stomach of a patient to a predetermined volume, wherein the device comprises a proximal end portion that is configured to be attached to a body cavity wall, and a distal end portion.

62. The device of claim 61, wherein the housing is configured to extend from the lower esophageal sphincter (LES) to the pyloric sphincter (PS).

63. The device of claim 61, wherein the housing is configured to extend from the lower esophageal sphincter (LES) to a location above the pyloric sphincter (PS).

64. The device of claim 61, wherein the housing comprises one or more openings therein that permit the passage of arteries and/or veins therethrough.

65. The device of claim 61, further comprising a sizing balloon positioned within the housing, wherein the sizing balloon is expandable and retractable to facilitate fitting of the housing to a stomach of a patient.

66. The device of claim 61, wherein the size of the distal end portion is transabdominally adjustable mechanically, magnetically, pneumatically, hydraulically, and/or telemetrically via RF energy or ultrasound.

67. A method of treating obesity, comprising wrapping a flexible housing around a stomach of a patient, wherein the housing restrains the stomach to a predetermined volume.

68. The method of claim 67, wherein the housing comprises a proximal end portion and a distal end portion, and further comprising attaching the proximal end portion to a body cavity wall.

69. The method of claim 67, wherein the housing comprises an inflatable sizing balloon disposed therein, and further comprising inflating the sizing balloon to facilitate fitting the housing to the stomach.

70. The method of claim 68, wherein the size of the distal end portion of the housing is adjustable around the stomach, and further comprising adjusting the size of the distal end portion.

71. The device of claim 70, wherein the size of the distal end portion of the housing is transabdominally adjustable, and wherein adjusting the size of the distal end portion is performed transabdominally.

72. The device of claim 71, wherein adjusting the size of the distal end portion is performed mechanically, magnetically, pneumatically, hydraulically, and/or telemetrically via RF energy or ultrasound.

73. The method of claim 67, wherein the flexible housing is wrapped around a stomach via a laparoscopy tool, via an endoscope, and/or via a NOTES device inserted down the esophagus of a patient.
